(19) United States
(12) Patent Application Publication
(10) Pub. No.: US 2005/0137641 A1
(43) Pub. Date: Jun. 23, 2005

(54) User interface for cardiac rhythm management device programmer

(76) Inventors: Alexandra Naughton, Minneapolis, MN (US); David Jeffrey, Ham Lake, MN (US)

Correspondence Address: Merchant & Gould P.C., P.O. Box 2903, Minneapolis, MN, 55402-0903, US

(21) Appl. No.: 10/731,422

(22) Filed: Dec. 8, 2003

(51) Int. Cl. A61N 1/362

(52) U.S. Cl. 607/32

(57) ABSTRACT

A user interface for a cardiac rhythm management device programmer. A plurality of cardiac rhythm management device families can be listed according to device family or according to specific model names and/or model numbers. In addition, high-level information related to specific cardiac rhythm management devices can be listed by a second module. The second module can accessed through selection of a specific device family or model name and/or model number listed by the first module.

### TECHNICAL FIELD

The present invention is related to user interfaces and, more particularly, to user interfaces for cardiac rhythm management device programmers.

### BACKGROUND

Cardiac rhythm management (“CRM”) devices are implanted in patients with heart abnormalities to assist in maintaining regular cardiac rhythms. CRM devices are known in the art and can be used to treat everything from bradycardia and tachycardia to degradation of the heart associated with congestive heart failure. See, e.g., U.S. Pat. Nos. 6,400,986, 6,427,084, and 6,622,040.

A caregiver can use a CRM device programmer (“programmer”) to communicate with and analyze data from CRM devices. For example, a programmer can be used to extract data stored in a CRM device related to the functioning of a patient's heart. In addition, a programmer can be used to reprogram a CRM device to provide new or different functionality to enhance a patient's condition.

A programmer typically provides a user interface to allow a caregiver to easily communicate with and program a CRM device. See, e.g., U.S. Pat. No. 6,353,761 to Conley et al., which is hereby incorporated by reference in its entirety. Upon initiation of the programmer during implantation, follow-up, and patient data-disk review, the caregiver is typically required to select the software associated with a given CRM device prior to that software being loaded by the programmer. However, because there are numerous CRM device manufacturers, as well as a variety of different model names and numbers associated with each CRM device of each manufacturer, it can be difficult for the caregiver to select the appropriate software on the programmer to review or analyze data from a specific CRM device.

Further, a caregiver may need additional high-level information associated with a specific CRM device during, for example, implantation or follow-up. This high-level information can include, for example, a CRM device's connector size or elective replacement indicator (“ERI”). This information is typically provided to the caregiver in paper form. See, e.g., Reference Guide to Pacemakers, ICDs and Leads, Compiled by Guidant CRM Technical Services, Guidant Corporation, March 2003. However, such information may not be readily accessible, should, for example, the paper form of the information not be available or be outdated. In addition, such high-level information is typically not accessible from the programmer.

Therefore, there is a need for improved user interfaces for cardiac rhythm management device programmers.

### SUMMARY

The present invention is related to user interfaces and, more particularly, to interfaces for cardiac rhythm management device programmers.

In example embodiments of a user interface, a plurality of cardiac rhythm management device families can be listed by, for example, a first module. The devices can be listed by the first module according to device family or according to specific model names and/or model numbers.

In addition, high-level information related to specific cardiac rhythm management devices can be listed by, for example, a second module. The second module can accessed through selection of a specific device family or model name and/or model number listed by the first module.

One aspect of the invention relates to a user interface for a programmer for a cardiac rhythm management system device. The user interface includes a first module listing a plurality of cardiac rhythm management system device families, and a plurality of second modules, each associated with one of the cardiac rhythm management system device families listed by the first module. The second module provides a plurality of high-level parameters associated with each of the cardiac rhythm management device families listed by the first module.

Another aspect of the invention relates to a user interface provided at startup of a programmer for a cardiac rhythm management system device, including a first module listing a plurality of cardiac rhythm management system device families, and a plurality of second modules, each associated with one of the cardiac rhythm management system device families and accessible through selection of one of the cardiac rhythm management system device families listed by the first module. The second module for each cardiac rhythm management system device family lists each cardiac rhythm management system device in a given cardiac rhythm management system device family by at least model name and model number, and the second module for each cardiac rhythm management system device family further lists at least one high-level parameter associated with each listed cardiac rhythm management system device selected from the group consisting of NBG code, x-ray identifier, connector size, polarity, defibrillator waveform, wrench, and elective replacement indicator. Interrogation/programming software associated with each cardiac rhythm management system device family is accessible by selecting a specific cardiac rhythm management system device family listed by the first module.

Yet another aspect of the invention relates to a cardiac rhythm management system, including a programmer for communicating with a cardiac rhythm management device, and a user interface for the programmer. The user interface includes a first module listing a plurality of cardiac rhythm management system device families, and a plurality of second modules, each associated with one of the cardiac rhythm management system device families listed by the first module, the second module providing a plurality of high-level parameters associated with each of the cardiac rhythm management device families listed by the first module.

Another aspect of the invention relates to method for providing a user interface for a programmer of a cardiac rhythm management system device, the method including: initializing the programmer, displaying an initial screen listing a plurality of cardiac rhythm management system device families, and displaying an information screen upon selection of a specific cardiac rhythm management system device family, the information screen providing a plurality of high-level parameters associated with cardiac rhythm management system devices of the specific cardiac rhythm management system device family.

### DESCRIPTION OF THE DRAWINGS

Reference will now be made to the accompanying drawings, which are not necessarily drawn to scale.

FIG. 1 is a schematic drawing illustrating one embodiment of portions of a cardiac rhythm management system and an environment in which the system is used.

FIG. 2 is a schematic drawing illustrating one embodiment of a cardiac rhythm management device coupled by leads to a heart of a patient.

FIG. 3 is a schematic drawing illustrating one embodiment of a cardiac rhythm management device programmer.

FIG. 4 illustrates one embodiment of a user interface for a cardiac rhythm management device programmer.

FIG. 5 illustrates the user interface of FIG. 4 with a listing of cardiac rhythm management device families provided.

FIG. 6 illustrates the user interface of FIG. 5 with a listing of information related to a specific cardiac rhythm management device family provided.

FIG. 7 illustrates another embodiment of a user interface for a cardiac rhythm management device programmer.

FIG. 8 is one embodiment of a flow diagram for a user interface of a cardiac rhythm management device programmer.

FIG. 9 illustrates another embodiment of a user interface for a cardiac rhythm management device programmer.

### DETAILED DESCRIPTION

Various embodiments of the present invention will be described in detail with reference to the drawings, wherein like reference numerals represent like parts and assemblies throughout the drawings. Reference to various embodiments does not limit the scope of the present invention, which is limited only by the scope of the claims attached hereto.

As used herein, a cardiac rhythm management (“CRM”) device is any device associated with a patient's heart that monitors the heart and/or provides therapy to the heart. Examples of such CRM devices include, without limitation, defibrillators (tachycardia devices), pacemakers (bradycardia devices), and cardiac resynchronization therapy devices (congestive heart failure devices), and components thereof (e.g., leads).

The present invention is related to user interfaces for CRM device programmers. Embodiments of user interfaces for CRM device programmers disclosed herein can be used to select an appropriate software package to be loaded by the programmer. In addition, the user interfaces can be used to provide high-level information associated with various CRM devices.

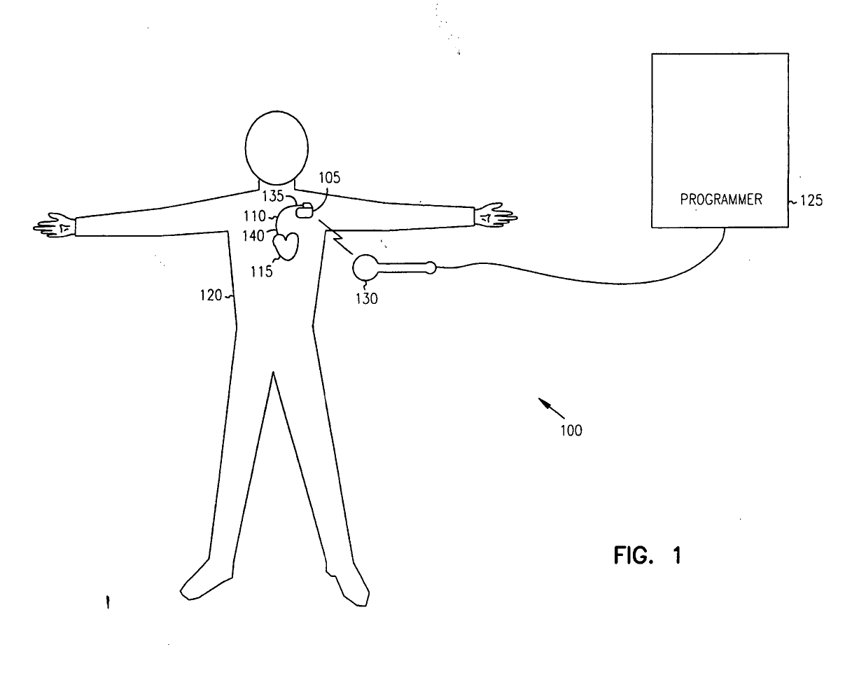

Referring now to FIG. 1, a schematic drawing illustrating generally and without limitation one embodiment of portions of a CRM system 100 and an environment in which the system 100 is used is shown. In FIG. 1, system 100 includes an implantable CRM device 105 that is coupled by a lead 110 (such as, for example, an intravascular endocardial lead) to a heart 115 of a patient 120. System 100 also includes an example CRM device programmer 125, which can wireless communicate with device 105 using a communication module such as telemetry device 130. Lead 110 includes a proximal end 135, which is coupled to CRM device 105, and a distal end 140, which is coupled to one or more portions of heart 115.

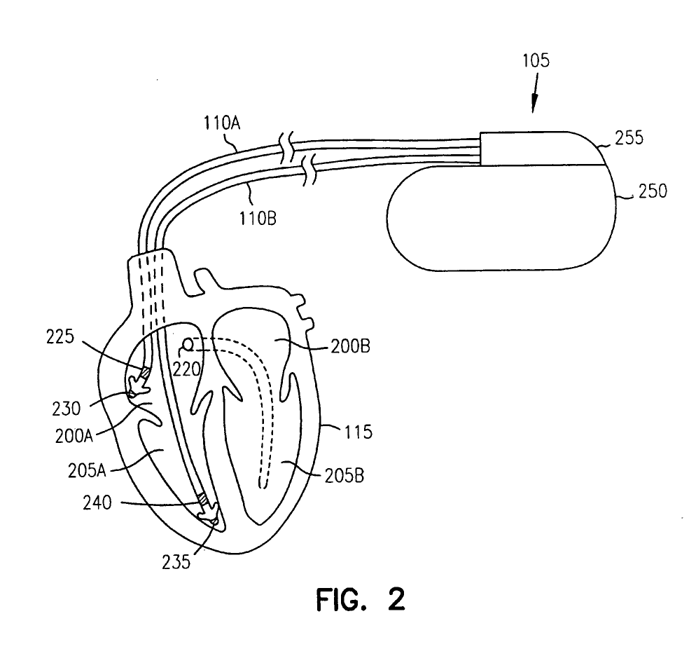

FIG. 2 is a schematic drawing illustrating generally and without limitation one embodiment of CRM device 105 coupled by leads 110A-B to heart 115, which includes a right atrium 200A, a left atrium 200B, a right ventricle 205A, a left ventricle 205B, and a coronary sinus 220 extending from right atrium 200A. In this embodiment, atrial lead 110A includes electrodes (electrical contacts) disposed in, around, or near an atrium 200 of heart 115, such as ring electrode 225 and tip electrode 230, for sensing signals and/or delivering pacing therapy to the atrium 200. Lead 110A optionally also includes additional electrodes, such as for delivering atrial and/or ventricular cardioversion/defibrillation and/or pacing therapy to heart 115.

In FIG. 2, a ventricular lead 110B includes one or more electrodes, such as tip electrode 235 and ring electrode 240, for delivering sensing signals and/or delivering pacing therapy. Lead 110B can optionally also include additional electrodes, such as for delivering atrial and/or ventricular cardioversion/defibrillation and/or pacing therapy to heart 115. Device 105 includes components that are enclosed in a hermetically-sealed can 250. Additional electrodes can be located on the can 250, or on an insulating header 255, or on other portions of device 105, for providing unipolar pacing and/or defibrillation energy in conjunction with the electrodes disposed on or around heart 115.

In one alternative embodiment, one of atrial lead 110A or ventricular lead 110B is omitted, thereby providing a “single chamber” device, rather than the dual chamber device illustrated in FIG. 2. In another embodiment, additional leads are provided for coupling device 105 to other heart chambers and/or other locations in the same heart chamber as one or more of leads 110A-B. The present method and apparatus will work in a variety of configurations and with a variety of electrical contacts or “electrodes.” Therefore, the arrangement illustrated in FIGS. 1 and 2 is provided by way of example only, and should not be construed as limiting the scope of the invention.

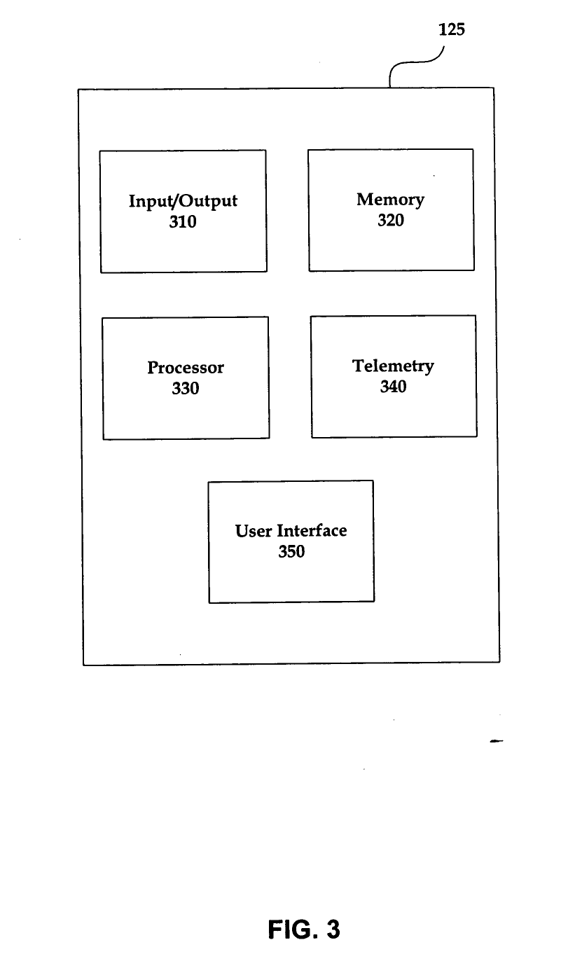

Referring now to FIG. 3, the example CRM device programmer 125 is illustrated schematically in greater detail. As noted above, the programmer 125 allows a caregiver to communicate with an implanted CRM device, such as device 105. The programmer 125 generally includes an input/output module 310, a memory module 320, a processor 330, a telemetry module 340, and a user interface module 350. It should be understood that programmer 125 is provided by way or example only, and that additional modules and/or functionality can also be included.

The input/output module 310 of programmer 125 allows the programmer 125 to communicate with external devices. For example, the input/output module 310 can include a disk drive (e.g., floppy, CD-ROM, DVD) that can read and write to removable storage media. In this manner, the input/output module 310 can read data from and write data to devices external to the programmer 125.

The memory module 320 is any conventional memory (volatile or non-volatile) that can be used to store data. The processor 330 is any conventional processor that can process data to, for example, analyze and present data communicated from the implanted CRM device 105. The telemetry module 340 includes components necessary to, for example, accomplish communication wirelessly between the programmer 125 and the implanted CRM device 105.

The user interface module 350 allows a user, such as the caregiver, to interact with the programmer 125 and receive information from the programmer 125. The user interface 350 can include, for example, software-driven screens that allow the caregiver to review and manipulate data communicated between the programmer 125 and the CRM device 105.

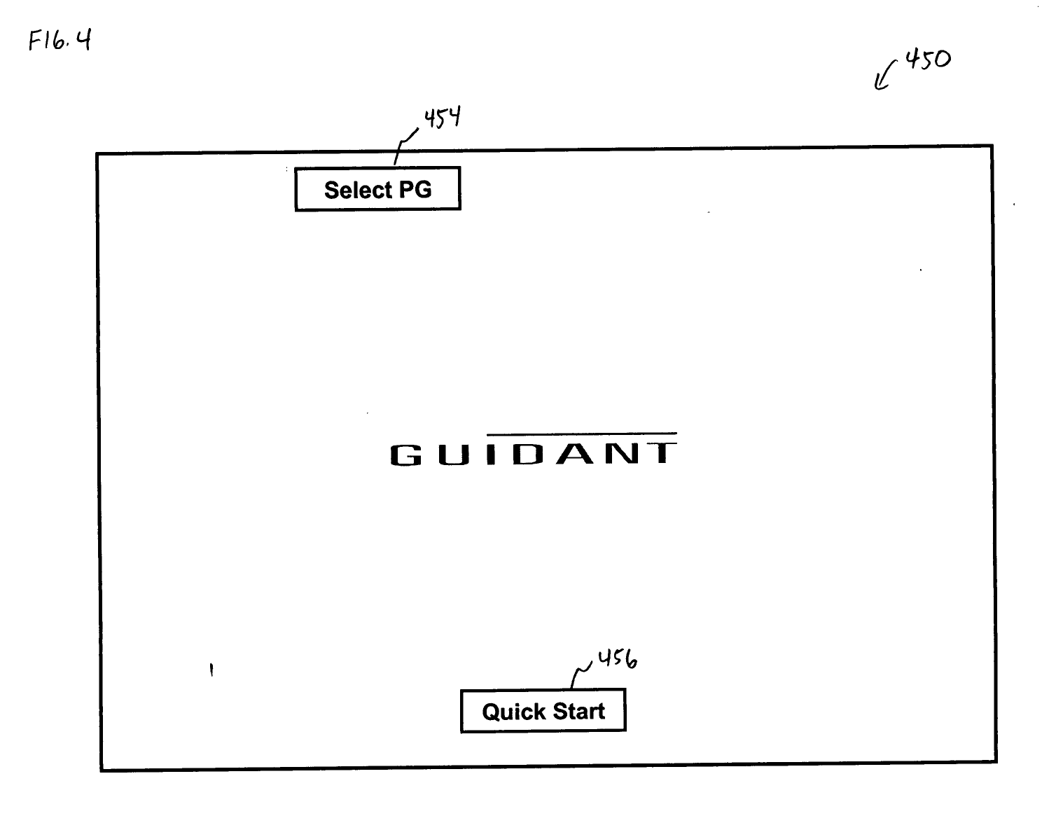

Referring now to FIGS. 4-6, one embodiment of an example user interface 450 for a programmer such as programmer 125 is illustrated. In the illustrated embodiment, the user interface 450 is software-driven and is displayed on a monitor to allow the caregiver to interact with the interface.

As shown in FIG. 4, the user interface 450 includes several buttons, including selection button 454 and quick start button 456.

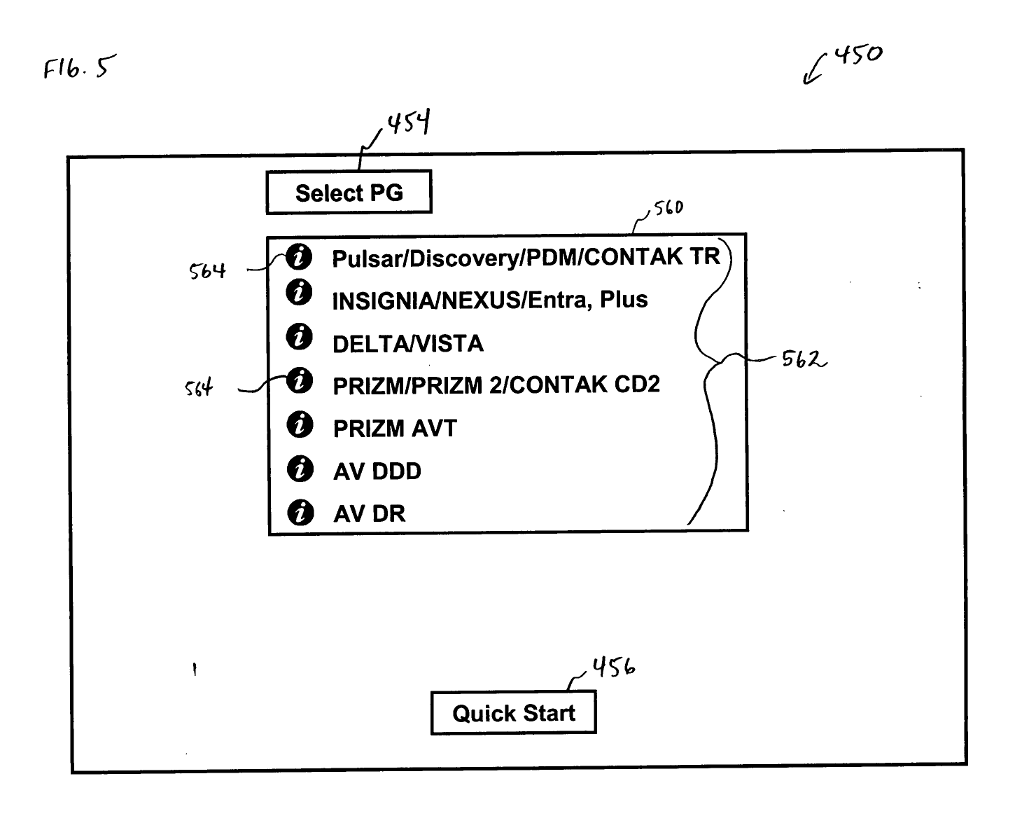

The quick start button 456 allows the programmer 125 to automatically identify the CRM device model during implantation and follow up. The selection button 454 allows a user to access a first module 560 described with reference to FIG. 5 below. Buttons 454 and 456 can be selected by the user in a variety of ways using, for example and without limitation, an input device such as a computer mouse, keyboard, or touch-screen apparatus.

Referring now to FIG. 5, the first module 560 (accessed through selection of selection button 454 as noted above) is illustrated. The first module 560 preferably includes a list of a plurality of CRM device families 562. For example and without limitation, one CRM device family listed by the first module 560 is Guidant Corporation's Pulsar/Discovery/PDM/CONTAK TR family of CRM devices. The list of CRM device families can include families from single or multiple CRM device manufacturers.

Preferably, each CRM device family 562 listed by the first module 560 is selectable using an input device. Once a CRM device family 562 is selected, the programmer 125 can load software associated with that specific device family to allow, for example, the programmer 125 to communicate with that specific type of CRM device, or to analyze data collected from that specific type of CRM device.

Also included, preferably for one or more of the device families 562 listed by the first module 560, is an information button 564. In the illustrated embodiment, an information button 564 is provided for each listed device family 562. If selected, the information button 564 provides access to high-level information specific to the selected device family. For example, selection of the information button 564 associated with the PRIZM/PRIZM 2/CONTAK CD2 CRM device family 562 listed by first module 560 provides access to a second module 670 including high-level information, as described further below.

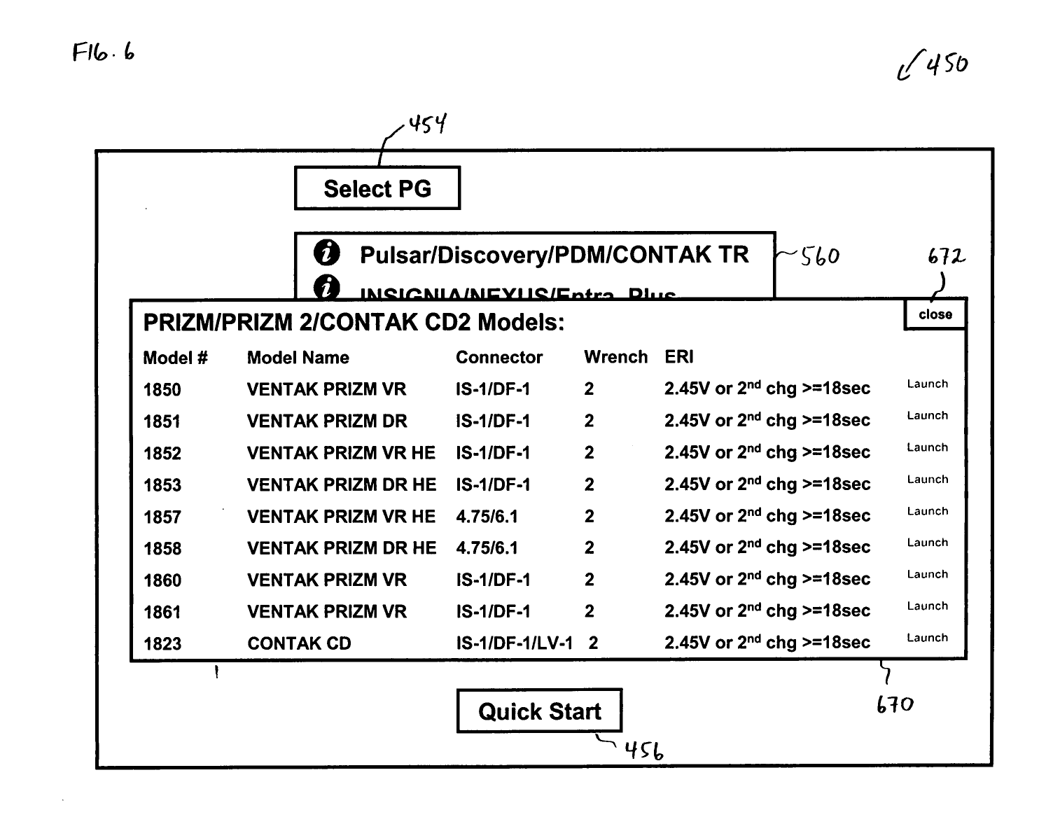

Referring now to FIG. 6, the second module 670 (accessed through selection of the information button 564 associated with a particular device family 562 listed by first module 560) is illustrated. The second module 670 preferably provides high-level information specific to different models of CRM devices in the CRM device family 562 selected from the first module 560.

For example and without limitation, the second module 670 can provide part or all of the following high-level information regarding each CRM device: model number; model name; connector; wrench; and elective replacement indicator (“ERI”). Although not shown, the second module 670 can also provide other high-level information relating to each CRM device such as, for example: NBG Code; NBD Code; X-Ray ID; connector location/placement, polarity, size, fixation, insulation, and length; beginning end of life (“BOL”)/ERI rate; defibrillator waveform; and BOL/ERI MAG rate. All of these parameters associated with CRM devices are well known in the art and will not be described in detail herein. Further, the example list of information provided above and shown in second module 670 is not exhaustive and should not be construed as limiting, as additional or other high-level information related to a specific CRM device can also be provided.

The high-level information provided by second module 670 can be presented in a tabular format as shown in FIG. 6, or in other similar formats. If the list of information provided by the second module 670 related to each CRM device is too long to fit on user interface 450, scrolling can be used to allow the user to scroll and locate the desired information. In addition, a close button 672 can be provided by the second module 670 to allow the user to close the second module 670 when desired to, for example, access information related to another CRM device family listed by first module 560.

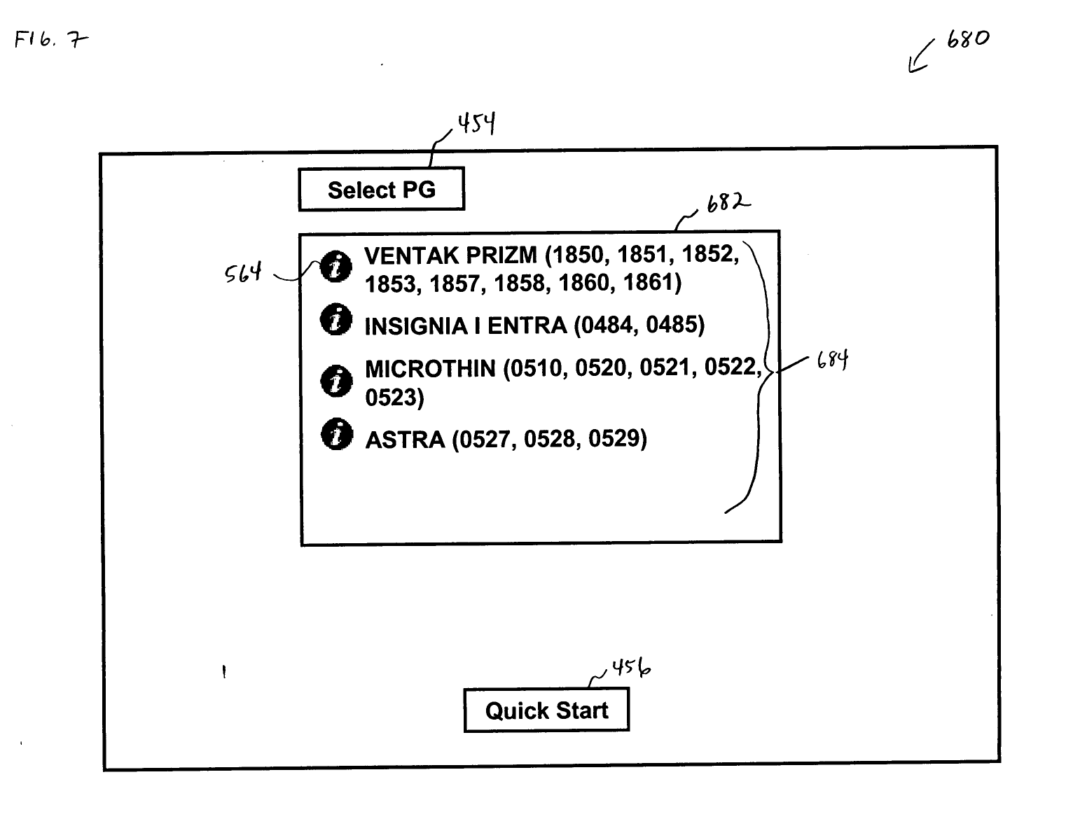

Referring now to FIG. 7, an alternative embodiment of a user interface 680 is illustrated. The interface 680 is similar to that of interface 450, except that the first module 682 includes a listing 684 of CRM devices by model name and model number, rather than by CRM device family. In another alternative embodiment, the user can select whether the CRM devices are listed by the first module 682 by CRM device family or by CRM device name and model number. In yet another embodiment, the CRM devices can be listed by both family name and device name/model number.

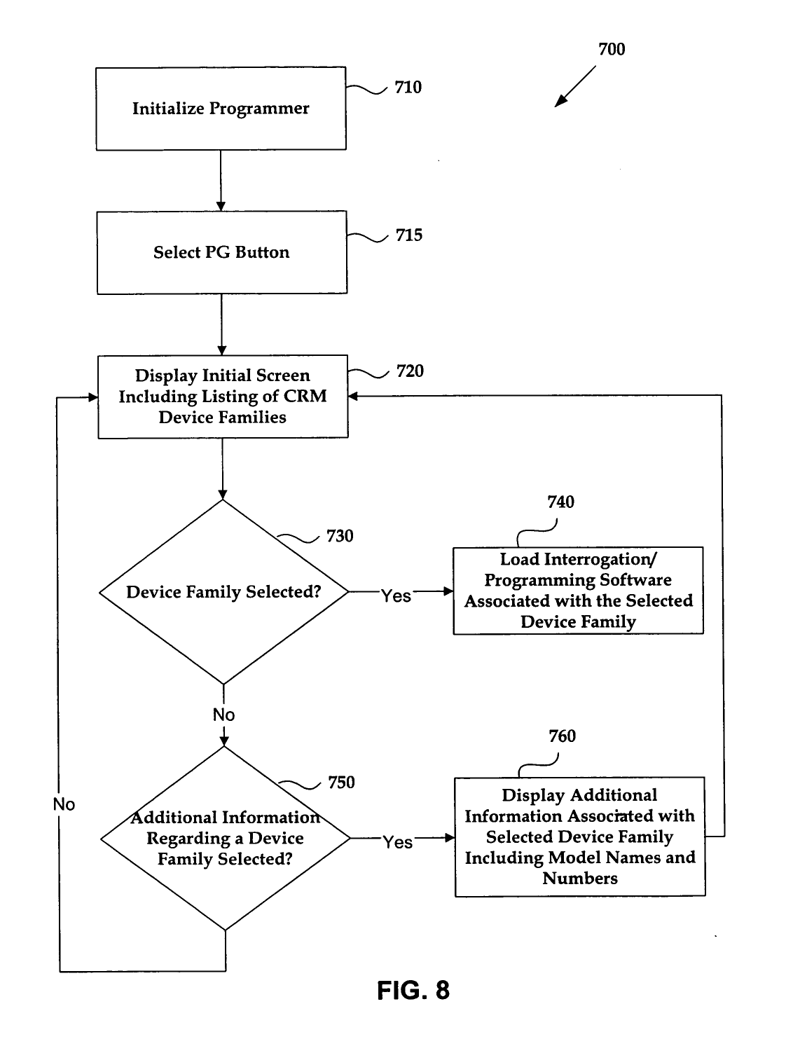

Referring now to FIG. 8, an example method 700 for using the user interface 450 is shown. The programmer is initialized at 710. This initialization can include powering on the programmer or resetting the programmer. At initialization, the user interface (such as interface 450) is provided. Next, the user selects the “Select PG” button to access the first module at 715. Upon selection, the interface provides a listing of CRM device families (e.g., first module 560) at 720.

Next, the user can use the interface to make desired selections. For example, the programmer determines whether the user has selected a specific device family at 730. If a device family has been selected, the programmer loads interrogation/programming software associated with that specific device family at 740. Alternatively, the programmer determines whether the user has selected the information button associated with a specific device family at 750. If the information button for a specific device family has been selected, the programmer displays high-level information associated with different models of the selected device family (e.g., second module 670) at 760. Once the user has closed the additional information, control can be passed back to the initial screen at 720.

The high-level information can be updated periodically. For example, the high-level information can be updated by inserting a removable storage media, including the updated high-level information, into the input/output module 310. In other embodiments, the high-level information can be updated by downloading the information from an update site through, for example, the Internet. In some embodiments, the programmer can be configured to automatically download updates a periodic intervals, thereby assuring that the high-level information is current.

There can be many advantages to a user interface configured as described herein. For example, providing the user interface upon initialization of the programmer allows the user to easily access high-level information (e.g., connector type, ERI) related to specific CRM device models by simply selecting an information button associated with a specific CRM device family. Such high-level information is thereby made readily available, and can be easily updated electronically at periodic or manually-selected intervals to assure that the most current information is available.

In addition, the user can easily select the appropriate software to be loaded by the programmer based on device family and/or based on device model name and model number. This can be advantageous, for example, during data-disk review by the caregiver, where only device name and/or model number may be provided to the caregiver.

Many alternative designs for the user interface disclosed herein can be provided. For example, the information provided by the user interface can be displayed in various manners. For example, instead of providing separate windows for the first and second modules, the information could be provided in a nested fashion. For example, a hierarchical nested structure from device family to device model to high-level information about a model can be provided.

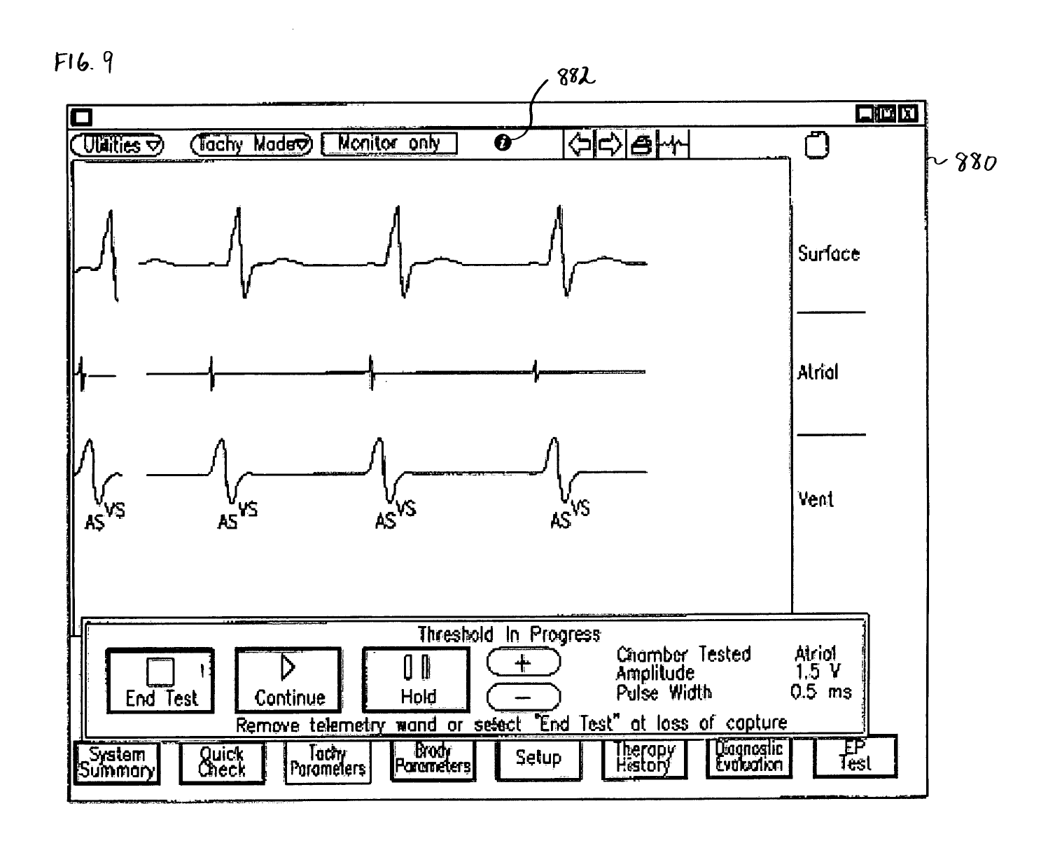

In addition, although it is preferable to provide the information associated with the user interface upon initialization of the programmer, the information can also be provided once software associated with a specific device family has been loaded by the programmer. For example, as shown in the alternative embodiment illustrated in FIG. 9, the example user interface 880 is generated by software loaded by the programmer for a specific CRM device family. An information button 882 can be provided on the user interface 880 to provide access to, for example, high-level information (e.g., second module 670) related to the CRM device.

The logical operations for implementing the user interfaces disclosed herein may be performed by a device other than a CRM device programmer. For example, a computer without telemetry capabilities can be used to perform data-disk review. Furthermore, the logical operations may be implemented (1) as a sequence of computer implemented steps running on a computer system, and/or (2) as interconnected machine modules.

This implementation is a matter of choice dependent on the performance requirements of the user interfaces. Accordingly, the logical operations making up the embodiments of the invention described herein are referred to as operations, steps, or modules. It will be recognized by one of ordinary skill in the art that the operations, steps, and modules may be implemented in software, in firmware, in special purpose digital logic, analog circuits, and any combination thereof without deviating from the spirit and scope of the present invention as recited within the claims attached hereto.

While the invention has been particularly shown and described with reference to preferred embodiments thereof, it will be understood by those skilled in the art that various other changes in the form and details may be made therein without departing from the spirit and scope of the invention.

1. A user interface for a programmer for a cardiac rhythm management system device, comprising:
a first module listing a plurality of cardiac rhythm management system device families; and
a plurality of second modules, each associated with one of the cardiac rhythm management system device families listed by the first module;
wherein the second module provides a plurality of high-level parameters associated with each of the cardiac rhythm management device families listed by the first module.

2. The user interface of claim 1, wherein the plurality of high-level parameters provided by the second module comprises at least one parameter selected from the group consisting of NBG code, x-ray identifier, connector size, polarity, defibrillator waveform, wrench, and elective replacement indicator.

3. The user interface of claim 1, wherein each second module is accessible through selection of one of the plurality of cardiac rhythm management system device families listed by the first module.

4. The user interface of claim 1, wherein interrogation/programming software associated with each cardiac rhythm management system device family is accessible by selecting a specific cardiac rhythm management system device family listed by the first module.

5. The user interface of claim 1, wherein the second module for each cardiac rhythm management system device family is accessible by selecting an information icon associated with each cardiac rhythm management system device family.

6. The user interface of claim 1, wherein the second module further lists each cardiac rhythm management system device in a given cardiac rhythm management system device family by at least model name and model number.

7. The user interface of claim 1, wherein the first module further lists each cardiac rhythm management device model name and model number for each listed cardiac rhythm management device family.

8. The user interface of claim 1, wherein the user interface is provided at startup of the programmer.

9. A user interface provided at startup of a programmer for a cardiac rhythm management system device, comprising:
a first module listing a plurality of cardiac rhythm management system device families; and
a plurality of second modules, each associated with one of the cardiac rhythm management system device families and accessible through selection of one of the cardiac rhythm management system device families listed by the first module;
wherein the second module for each cardiac rhythm management system device family lists each cardiac rhythm management system device in a given cardiac rhythm management system device family by at least model name and model number;
wherein the second module for each cardiac rhythm management system device family further lists at least one high-level parameter associated with each listed cardiac rhythm management system device selected from the group consisting of NBG code, x-ray identifier, connector size, polarity, defibrillator waveform, wrench, and elective replacement indicator; and
wherein interrogation/programming software associated with each cardiac rhythm management system device family is accessible by selecting a specific cardiac rhythm management system device family listed by the first module.

10. The user interface of claim 9, wherein the second module for each cardiac rhythm management system device family is accessible by selecting an information icon associated with each cardiac rhythm management system device family provided by the first module.

11. A cardiac rhythm management system, comprising:
a programmer for communicating with a cardiac rhythm management device; and
a user interface for the programmer, including:
a first module listing a plurality of cardiac rhythm management system device families; and
a plurality of second modules, each associated with one of the cardiac rhythm management system device families listed by the first module;
wherein the second module provides a plurality of high-level parameters associated with each of the cardiac rhythm management device families listed by the first module.

12. The system of claim 11, wherein the plurality of high-level parameters comprises at least one parameter selected from the group consisting of NBG code, x-ray identifier, connector size, polarity, defibrillator waveform, wrench, and elective replacement indicator.

13. The system of claim 11, wherein each second module of the user interface is accessible through selection of one of the plurality of cardiac rhythm management system device families listed by the first module.

14. The system of claim 11, wherein interrogation/programming software associated with each cardiac rhythm management system device family is accessible by selecting a specific cardiac rhythm management system device family listed by the first module.

15. A method for providing a user interface for a programmer of a cardiac rhythm management system device, the method comprising:
initializing the programmer;
displaying an initial screen listing a plurality of cardiac rhythm management system device families; and
displaying an information screen upon selection of a specific cardiac rhythm management system device family, the information screen providing a plurality of high-level parameters associated with cardiac rhythm management system devices of the specific cardiac rhythm management system device family.

16. The method of claim 15, wherein the step of displaying the information screen further comprises displaying information selected from the group consisting of NBG code, x-ray identifier, connector size, polarity, defibrillator waveform, wrench, and elective replacement indicator as the plurality of parameters.

17. The method of claim 15, further comprising loading interrogation/programming software associated with a specific cardiac rhythm management system device family upon selection of the specific cardiac rhythm management system device family.
